(19) United States
(12) Patent Application Publication
(10) Pub. No.: US 2023/0404624 A1
(43) Pub. Date: Dec. 21, 2023

(54) PATIENT-SPECIFIC SPINAL ROD USING 3D PRINTING AND PEDICLE FIXATION DEVICE INCLUDING THE SAME

(71) Applicant: MEDYSSEY CO., LTD., Chungcheongbuk-do (KR)

(72) Inventors: Su Heon WOO, Gyeonggi-do (KR); Hyun Woo JUNG, Chungcheongbuk-do (KR); Jin Won KIM, Chungcheongbuk-do (KR)

(73) Assignee: MEDYSSEY CO., LTD., Chungcheongbuk-do (KR)

(21) Appl. No.: 18/329,124

(22) Filed: Jun. 5, 2023

(30) Foreign Application Priority Data
Jun. 15, 2022 (KR) 10-2022-0072608

(51) Int. Cl. A61B 17/70; A61B 34/10; B33Y 80/00

(52) U.S. Cl. CPC A61B 17/7011; A61B 2017/568; B33Y 80/00; A61B 34/10

(57) ABSTRACT

Proposed is a patient-specific spinal rod using 3D printing and a pedicle fixation device including the same. The rod is characterized in that information of a damaged spine is obtained through X-rays or computed tomography (CT) scans before surgery, an image is created by considering the setting of a coupling part of the rod that is combined with a pedicle screw inserted and fixed in a vertebral body to allow two or more vertebral bodies to fuse and a bending shape according to curve of a spine, and a metal material is melted and output by 3D printing to have an appearance corresponding to the created image. The rod includes a rod-shaped coupling portion coupled with the pedicle screw, and a support portion configured to extend in a longitudinal direction of the coupling portion to serve as a support for holding the spine.

### CROSS REFERENCE TO RELATED APPLICATION

The present application claims priority to Korean Patent Application No. 10-2022-0072608, filed Jun. 15, 2022, the entire contents of which is incorporated herein for all purposes by this reference.

### BACKGROUND OF THE INVENTION

### Field of the Invention

The present disclosure relates to a patient-specific spinal rod using 3D printing and a pedicle fixation device including the same and, more particularly, to a patient-specific spinal rod using 3D printing in which coupling parts of the rod that are combined with pedicle screws are set on the basis of X-ray or computed tomography (CT) images before surgery, and that greatly improves the performance of supporting the spine by increasing the diameter in the non-coupling part.

### Description of the Related Art

In general, spinal stenosis is a condition that occurs when the spinal canal, a passage through which the spinal nerves pass, is narrowed due to degenerative factors, etc., which results in pressure on the nerves in all directions, causing various neurological abnormalities such as neurogenic intermittent claudication of the nerve roots. Spinal stenosis is common in elderly.

When the problem caused by spinal stenosis is only nerve compression, removing the affected bone and ligament tissue in which a nerve or group of nerves are compressed. However, when there is back pain and segmental instability due to nerve compression and degenerative changes (disc degeneration, facet joint degeneration), spinal fusion is required.

In a typical spinal fusion surgery, after removing the intervertebral disc between the affected vertebrae, a hollow metal or plastic artificial aid (cage) packed with bone chips is inserted into the area from which the disc was removed, then pedicle screws are inserted and fixed in the vertebral bodies above and below the damaged disc, and then a rod is used to connect the pedicle screws for fixation.

During spinal fusion, the rod plays the role of a support holding the spine in combination with the pedicle screws, so that the interbody fusion proceeds well.

As an example of related art, Korean Patent No. 10-0645377 “PEDICLE FIXATION DEVICE” discloses a rod in the form of an elastic rod or coil spring. Since the entire rod has elasticity, it can be curved according to the shape of the patient's spine. That is, the rod is curved along with the change in the shape of the spine due to the patient's movement and activity, and has a restoring force, so that comfort as well as treatment can be provided to the patient.

However, in the case of a rod having the shape of an elastic rod or coil spring, despite its excellent elasticity, there is a problem with the spring undesirably moving, and when the rod is fastened to the pedicle for a long period of time, the spring is deformed and does not maintain the shape of the spinal curve suitable for the human body.

In addition, since there is a difference in the bending of each part of the spine, when performing procedures on two or more vertebrae, it is necessary to differentiate and precisely consider the direction of bending forward and backward and the direction of bending left and right. Yet, conventional rods are difficult to apply to the spine with a precise structure to fit the curve of the spine, and medical staff have to bend and cut the rod during surgery to fit the curve of the spine, leading to a longer operation time.

Moreover, in the case of conventional rods, a rod manufactured with a uniform diameter is cut and used without having designated sites thereon for joining with pedicle screws. Because the thickness of the rod is constant, two rods should be overlapped and used for areas of the spine that need more support. That is, in order to strongly support the spine, there is no other way other than installing two overlapping rods, and thus, a number of addtional parts are required and the structure becomes complicated.

Furthermore, when it is necessary to protect the spinal cord, such as in laminectomy, in which the lamina of the spine is removed to enlarge the spinal canal, conventionally, a number of parts including a protective device are required, which has the disadvantage of taking a longer operation time. Therefore, a technology capable of solving these problems is needed.

### SUMMARY OF THE INVENTION

Accordingly, the present disclosure has been made keeping in mind the above problems occurring in the related art, and the present disclosure is intended to provide a patient-specific spinal rod using 3D printing in which coupling parts of the rod that are combined with pedicle screws are set in advance on the basis of preoperative X-ray or computed tomography (CT) images according to the curve of the patient's spine, and that greatly improves the performance of supporting the spine by increasing the diameter in the non-coupling part.

An objective of the present disclosure is to provide a patient-specific spinal rod using 3D printing that prevents unnecessary material loss as the rod is custom-made according to the required length and size before surgery.

An objective of the present disclosure is to provide a patient-specific spinal rod using 3D printing that does not require cutting and bending of the rod during surgery and reduces the number of parts and significantly shorten the operation time as the rod can be integrated with a protective device used in laminectomy, etc.

An objective of the present disclosure is to provide a pedicle fixation device that enables planned surgery and does not use a large number of parts, allowing surgery to be performed easily in a much shorter time compared to conventional spinal fusion, which significantly reduces the burden of surgery on both doctors and patients.

In order to achieve the above objective, according to an embodiment of the present disclosure, there is provided a patient-specific spinal rod using 3D printing that is characterized in that information of a damaged spine is obtained through X-rays or computed tomography (CT) scans before surgery, an image is created by considering the setting of a coupling part of the rod that is combined with a pedicle screw inserted and fixed in a vertebral body to allow two or more vertebral bodies to fuse and a bending shape according to a curve of a spine, and a metal material is melted and output by 3D printing to have an appearance corresponding to the created image, the rod including: a rod-shaped coupling portion coupled with the pedicle screw; and a support portion integrally formed with the coupling portion by extending in a longitudinal direction of the coupling portion, and configured to have a diameter or shape different from that of the coupling portion to serve as a support for holding the spine.

In the present disclosure, the coupling portion and the support portion may be formed in a three-dimensional shape of one of a lordosis curve, a kyphosis curve, and a scoliosis curve, or in which two or more of these curves are mixed according to the curve of the spine.

The support portion may be composed of at least two or more, and each support portion may have a different diameter or shape.

Particularly, the support portion may be a high-strength support portion for strongly supporting the spine.

In this case, the high-strength support portion may have a cylindrical rod shape with a larger diameter than the coupling portion, or may have a rectangular parallelepiped shape with a width or thickness greater than the diameter of the coupling portion.

Meanwhile, when the high-strength support portion is formed in a rectangular parallelepiped, the high-strength support portion may have a shape having a radius of curve that is gently curved in a transverse direction toward the spine.

In addition, the support portion may be an elastic support portion having elasticity so as to be bent along with a change in a shape of the spine due to a patient's movement and activity and to provide comfort and treatment.

In the present disclosure, a plurality of convex protrusions may be formed on an outer surface of the coupling portion to improve a fixing force by increasing a frictional force with the pedicle screw.

Meanwhile, the coupling portion and the support portion may be made of at least one of a titanium alloy (Gr23), a Ti—Ni-based alloy, stainless steel, a shape memory polymer, and a Co—Cr—Mo-based alloy.

In addition, in the present disclosure, at least one of a crosslink, a connector, or a spinal cord protector connecting two support portions in a transverse direction may be integrally formed with the rod.

In addition, in order to achieve the above objective, in the present disclosure, there is provided a pedicle fixation device, including: a pedicle screw provided with a head portion configured to have an accommodation groove of a predetermined depth formed thereon, and a screw portion configured to be driven into a bottom surface of the head portion by a predetermined depth in a vertical direction of a pedicle, and serves as a fixing base for a damaged pedicle; a rod seated in the accommodation groove, and configured to connect two or more pedicle screws to fix an angle and spacing of the pedicle; and a fixing means fastened to the accommodation groove of the pedicle screw and configured to pressurize the rod to prevent the rod from moving, wherein the rod may be characterized in that information of a damaged spine is obtained through X-rays or computed tomography (CT) scans before surgery, an image is created by considering the setting of a coupling part of the rod that is combined with a pedicle screw inserted and fixed in a vertebral body to allow two or more vertebral bodies of the damaged spine to fuse and a bending shape according to a curve of a spine, and a metal material is melted and output by 3D printing to have an appearance corresponding to the created image, and may include: a rod-shaped coupling portion coupled with the pedicle screw; and a support portion integrally formed with the coupling portion by extending in a longitudinal direction of the coupling portion, and configured to have a diameter or shape different from that of the coupling portion to serve as a support for holding the spine.

According to the present disclosure as described above, it is possible to provide a rod most suitable for the patient's spine shape by manufacturing and using a patient-specific spinal rod by 3D printing according to the curve of the patient's spine by utilizing preoperative X-ray or computed tomography (CT) images. In particular, it is possible to greatly improve the performance of supporting the spine by pre-setting coupling parts of the rod that are combined with pedicle screws and increasing the diameter in the non-coupling part.

Furthermore, it is possible to prevent unnecessary material loss as the rod is custom-made according to the required length and size before surgery, and reduce the number of parts and significantly shorten the operation time since cutting and bending of the rod during surgery are not required and the rod can be integrated with a protective device used in laminectomy, etc.

Furthermore, it is possible to significantly reduce the burden of surgery on both doctors and patients since a surgery can be planned and a large number of parts are not used, allowing surgery to be performed easily in a much shorter time compared to conventional spinal fusion.

### BRIEF DESCRIPTION OF THE DRAWINGS

The above and other objectives, features, and other advantages of the present disclosure will be more clearly understood from the following detailed description when taken in conjunction with the accompanying drawings, in which:

FIGS. 1 and 2 are perspective views showing a patient-specific spinal rod using 3D printing for spinal fusion according to an embodiment of the present disclosure;

FIG. 3 is a view showing an application example of the rod according to the embodiment of the present disclosure;

FIG. 4 is a perspective view showing a method of coupling the rod of the present disclosure and a pedicle screw;

FIG. 5 is a perspective view showing a method of coupling the rod of the present disclosure and a pedicle screw, a perspective view showing another example of the rod;

FIGS. 6A and 6B are perspective views showing that the rod of the present disclosure is integrally formed with a crosslink, a connector, or a spinal cord protector connecting in the transverse direction; and

FIG. 7 is a view showing an application example of the rod shown in 6A and 6B.

### DETAILED DESCRIPTION OF THE INVENTION

Advantages and features of the present disclosure, and methods for achieving them, will become clear with reference to the embodiments described below in detail in conjunction with the accompanying drawings. However, the present disclosure is not limited to the embodiments disclosed below and will be implemented in various different forms. The embodiments are provided only to complete the disclosure of the present disclosure, and to completely inform those skilled in the art of the scope of the invention to which the present disclosure pertains. The present disclosure is only defined by the scope of the claims. Like reference numbers designate like elements throughout the specification.

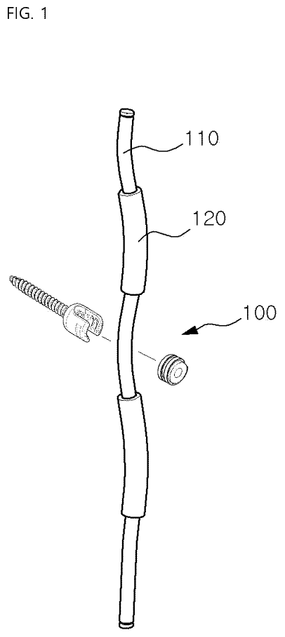

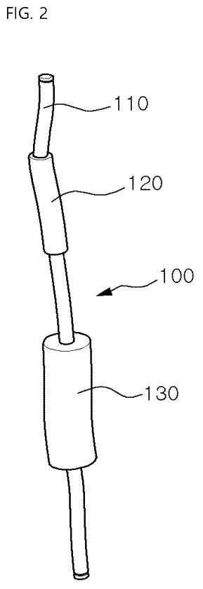

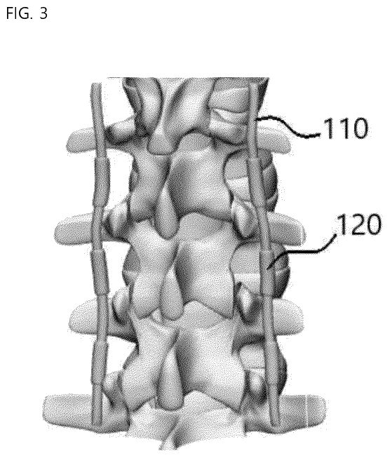

FIGS. 1 and 2 are perspective views showing a patient-specific spinal rod using 3D printing for spinal fusion according to an embodiment of the present disclosure, and FIG. 3 is a view showing an application example of the rod according to the embodiment of the present disclosure.

A patient-specific spinal rod 100 of the present disclosure is characterized in that information of a damaged spine through X-rays or computed tomography (CT) scans before surgery is obtained, an image of the rod 100 on the basis of the obtained information is created, and a material is melted and output by 3D printing to have an appearance corresponding to the created image.

In creating the image of the rod 100, after obtaining information of the damaged spine, on the basis of the obtained information, a coupling part of the rod that is combined with a pedicle screw inserted and fixed in the vertebral body is set, and considering a bending shape according to curve of the spine, the diameter in the non-coupling part is increased, so that the performance of supporting the spine is greatly improved.

The rod 100 may be made of metal. For example, the rod 100 may be made of at least one of a titanium alloy (Gr23), a Ti—Ni-based alloy, stainless steel, a shape memory polymer, and a Co—Cr—Mo-based alloy.

When the rod 100 is manufactured with a 3D printer, after melting a metal material, a process of melting and attaching the metal material in a layered manner using additive manufacturing, a method of producing an object identically to the created image, may be performed.

As shown in FIGS. 1 and 2, the rod 100 includes: a rod-shaped coupling portion 110, which is a part that is combined with the pedicle screw; and a support portion 120 configured to extend in the longitudinal direction of the coupling portion 110 to be integrally formed, and have a diameter or shape different from that of the coupling portion 110 to serve as a support for holding the spine.

In the present disclosure, the coupling portion 110 and the support portion 120 may be formed in a three-dimensional shape of one of a lordosis curve, a kyphosis curve, and a scoliosis curve, or in which two or more of these curves are mixed according to the curve of the spine.

That is, since the vertebral body into which the pedicle screw is inserted is different and the direction of curve of the spine is also different depending on the position of the spine where spinal fusion is performed, the coupling portion 110 and the support portion 120 may be formed in a three-dimensional shape in which two or more of the lordosis, kyphosis, and scoliosis are mixed according to the curve of the spine as shown in FIGS. 1 and 2. In the present disclosure, the shape of the spine is checked through X-rays or computed tomography (CT) scans before surgery, so that the lengths of the coupling portion 110 and the support portion 120 may be custom-made according to the shape of the patient's spine and the distance between the vertebrae.

As such, since the rod 100 is custom-made according to the required length and size, unnecessary material loss is prevented, and cutting and bending of the rod during surgery are not required.

The support portion 120 is integrally formed with the coupling portion 110 by extending in the longitudinal direction of the coupling portion 110, and has the diameter or shape different from that of the coupling portion 110 to serve as a support for holding the spine.

That is, because the coupling portion 110 is a portion coupled to a pedicle screw, the coupling portion 110 may be formed in a rod shape with a constant diameter, but portions other than the coupling portion 110 may need to strongly support the spine. To this end, it is preferable that the support portion 120 of the rod 100 has a cylindrical rod shape with a larger diameter than the coupling portion 110 so as to strongly support the spine.

Conventionally, for the lumber spine, a rod with a diameter of about 55 to 65 mm is used. However, in the case of the patient-specific spinal rod 100 of the present disclosure, the location of the coupling portion 110 coupled with a pedicle screw is checked through X-rays or computed tomography (CT) scans before surgery, and the diameter of the part other than the coupling portion 110 is increased in order to manufacture a high-strength rod.

At this time, the shape of the support portion 120 is not limited to a cylindrical bar shape, and the support portion 120 may be formed in a rectangular parallelepiped shape having a width or thickness greater than the diameter of the coupling portion 110 so as to reinforce the supporting force for supporting the spine as needed. For example, the support portion may be formed in a rectangular parallelepiped shape having a wide width to widely support the spine.

In this way, the support portion 120 may be a high-strength support portion for strongly supporting the spine, and when the high-strength support portion is formed in a rectangular parallelepiped, the high-strength support portion may have a shape having a radius of curve that is gently curved in a transverse direction toward the spine.

This is a shape in consideration of the contact surface that comes into contact with the spine because the support portion is formed in a shape surrounding the spine.

Meanwhile, the support portion 120 may be provided in plurality of two or more. At this time, individual support portions 120 and 130 may have a diameter or shape of a different size. That is, as shown in FIG. 2, the support portion 120 may consist of a first support portion 120 and a second support portion 130, and the second support portion 130 provided at a position where it is necessary to more strongly support the spine may have a larger diameter by increasing the diameter thereof.

At this time, as described above, the shape of the first support portion 120 or the second support portion 130 may be a shape other than a cylindrical rod.

FIG. 3 is a view showing an application example of the rod according to the embodiment of the present disclosure. The image of the rod 100 is created in consideration of a bending shape according to curve of a spine after obtaining information of the damaged spine through X-rays or computed tomography (CT) scans, and setting a coupling part of the rod that is combined with a pedicle screw inserted and fixed in a vertebral body on the basis of the obtained information.

At this time, in determining the length, thickness or shape of the coupling portion 110 and the support portion 120, an image is created according to the shape and curve of the spine, and then a material is melted and output by 3D printing to have an appearance corresponding to the created image.

Meanwhile, the support portion 120 may be an elastic support portion having elasticity so as to be bent along with the change in the shape of the spine due to the patient's movement and activity and to provide comfort and treatment.

The shape of the elastic support portion may be a leaf spring, or may be varied to obtain the required anteroposterior and left-right bending angles, so that the elastic support portion may be easily restored by restoring force without disturbing the patient's movement and activity as much as possible. In this case, it is preferable to properly adjust the length, thickness, and width of the elastic support portion to obtain a desired elastic force and a maximum bending angle.

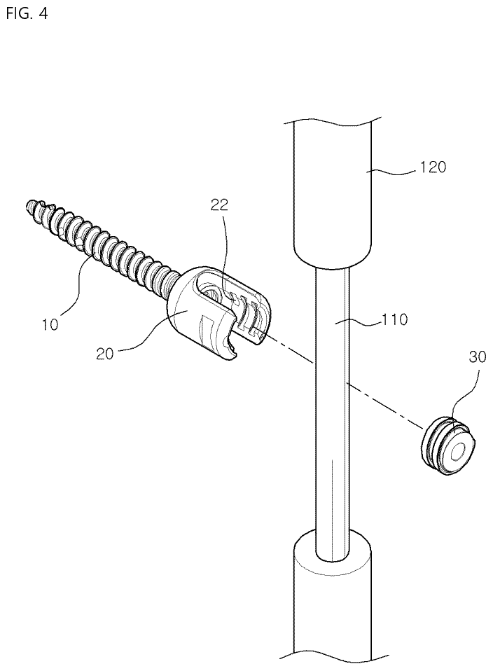

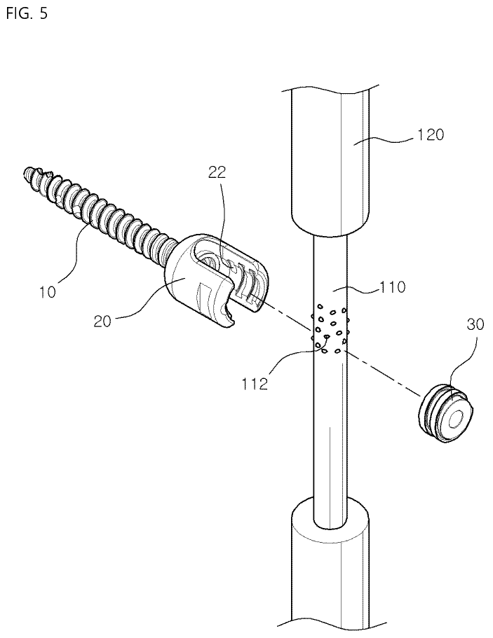

FIG. 4 is a perspective view showing a method of coupling the rod of the present disclosure and a pedicle screw, and FIG. 5 is a perspective view showing a method of coupling the rod of the present disclosure and a pedicle screw, a perspective view showing another example of the rod.

In the rod 100 of the present disclosure, the coupling portion 110 is coupled with a pedicle screw to fix the vertebral body. The pedicle screw includes a head portion 20 having an accommodation groove 22 of a predetermined depth formed thereon, and a screw portion 10 that is driven into the bottom surface of the head portion 20 by a predetermined depth in the vertical direction of the pedicle, and serves as a fixing base for a damaged pedicle. The coupling portion 110 of the rod 100 is seated in the accommodation groove 22, and the angle and spacing of the pedicle are fixed by connecting two or more pedicle screws, and a fixing means 30 for pressurizing the rod to prevent it from moving is fastened to the accommodation groove 22 of the pedicle screw.

A female screw is formed in the accommodation groove 22 and a male screw fastenable to the female screw is formed in the fixing means 30 so that the pedicle screw and the fixing means may be coupled by screw fastening.

In this way, the coupling portion 110 of the rod is inserted into the accommodation groove 22 of the pedicle screw, and the accommodation groove 22 and the fixing means 30 are screwed to fix the rod. Yet, the possibility that the fastening of the accommodation groove 22 and the fixing means 30 is gradually loosened over time cannot be ruled out.

Thus, a configuration may be required to more stably secure the state in which the rod is seated in the accommodation groove 22 of the pedicle screw.

To this end, in the present disclosure, a plurality of convex protrusions 112 are formed on the outer surface of the coupling portion 110 to improve the fixing force by increasing the frictional force with the pedicle screws. Due to the protrusions 112, the surface of the coupling portion 110 is not smooth, and accordingly, the fixation force may be improved by increasing the frictional force when combined with the pedicle screw.

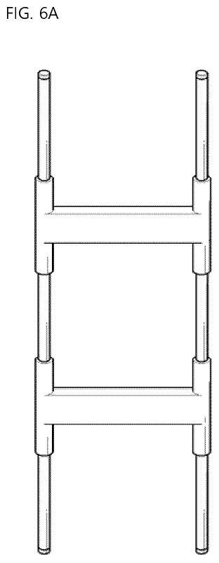

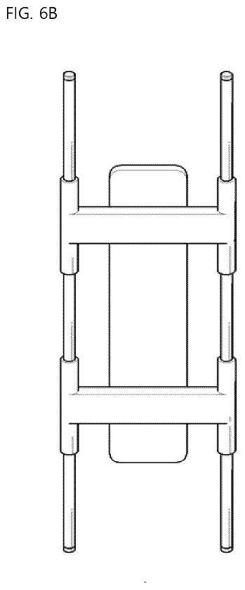

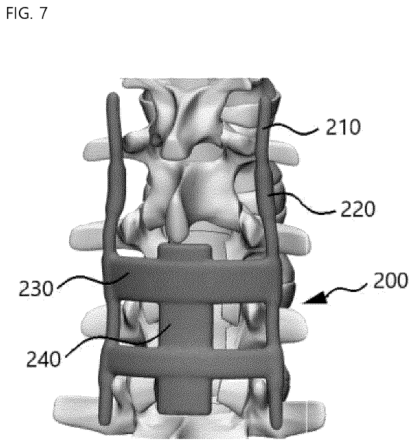

FIGS. 6A and 6B are perspective views showing that the rod of the present disclosure is integrally formed with a crosslink, a connector, or a spinal cord protector connecting in the transverse direction. FIG. 7 is a view showing an application example of the rod shown in 6A and 6B.

As shown in FIGS. 6A and 6B, in the present disclosure, a crosslink, a connector, or a spinal cord protector connecting the two support portions in the transverse direction may be integrally formed with the rod.

FIG. 6A shows that a crosslink connecting two support portions in the transverse direction is integrally formed with the rod, and FIG. 6A shows that a crosslink and a spinal cord protector are integrally formed with the rod. In the drawings, although the rod, the crosslink, and the spinal cord protector are shown in a standardized shape, in actual 3D printing, they are produced in a shape that is bent in consideration of curve according to the shape of the patient's spine.

Conventionally, a separate crosslink needs to be installed to connect two rods in the transverse direction, and a connector is installed to connect the rods in the longitudinal direction. In contrast, in the present disclosure, when manufacturing a rod, the rod may be manufactured by 3D printing so that the crosslink and connector are integrally formed.

Furthermore, conventionally, when it is necessary to protect the spinal cord, such as in laminectomy, in which the lamina is removed to open up the spinal canal, conventionally, an additional protective device needs to be installed. However, in the present disclosure, since a rod and a protective device used in laminectomy, etc. may be integrated, it is possible to reduce the number of parts and greatly shorten the operation time.

According to the patient-specific spinal rod using 3D printing of the present disclosure, it is possible to significantly reduce the burden of surgery on both doctors and patients since a surgery can be planned and a large number of parts are not used, allowing surgery to be performed easily in a much shorter time compared to conventional spinal fusion.

In the above, although the preferred embodiments of the present disclosure have been mainly described, various changes or modifications may be made at the level of a person skilled in the art in the technical field to which the present disclosure pertains. Such changes and modifications may be said to belong to the present disclosure as long as they do not deviate from the scope of the technical idea provided by the present disclosure. Therefore, the scope of the present disclosure will be defined by the claims described below.

What is claimed is:

1. A patient-specific spinal rod using 3D printing that is characterized in that information of a damaged spine is obtained through X-rays or computed tomography (CT) scans before surgery, an image is created by considering the setting of a coupling part of the rod that is combined with a pedicle screw inserted and fixed in a vertebral body to allow two or more vertebral bodies to fuse and a bending shape according to a curve of a spine, and a metal material is melted and output by 3D printing to have an appearance corresponding to the created image, the rod comprising:
a rod-shaped coupling portion coupled with the pedicle screw; and
a support portion integrally formed with the coupling portion by extending in a longitudinal direction of the coupling portion, and configured to have a diameter or shape different from that of the coupling portion to serve as a support for holding the spine.

2. The patient-specific spinal rod using 3D printing of claim 1, wherein the coupling portion and the support portion are formed in a three-dimensional shape of one of a lordosis curve, a kyphosis curve, and a scoliosis curve, or in which two or more of these curves are mixed according to the curve of the spine.

3. The patient-specific spinal rod using 3D printing of claim 1, wherein the support portion is composed of at least two or more, and each support portion has a different diameter or shape.

4. The patient-specific spinal rod using 3D printing of claim 1, wherein the support portion is a high-strength support portion for strongly supporting the spine.

5. The patient-specific spinal rod using 3D printing of claim 4, wherein the high-strength support portion has a cylindrical rod shape with a larger diameter than the coupling portion, or has a rectangular parallelepiped shape with a width or thickness greater than the diameter of the coupling portion.

6. The patient-specific spinal rod using 3D printing of claim 5, wherein when the high-strength support portion is formed in a rectangular parallelepiped, the high-strength support portion has a shape having a radius of curve that is gently curved in a transverse direction toward the spine.

7. The patient-specific spinal rod using 3D printing of claim 1, wherein the support portion is an elastic support portion having elasticity so as to be bent along with a change in a shape of the spine due to a patient's movement and activity and to provide comfort and treatment.

8. The patient-specific spinal rod using 3D printing of claim 1, wherein a plurality of convex protrusions are formed on an outer surface of the coupling portion to improve a fixing force by increasing a frictional force with the pedicle screw.

9. The patient-specific spinal rod using 3D printing of claim 1, wherein the coupling portion and the support portion are made of at least one of a titanium alloy (Gr23), a Ti—Ni-based alloy, stainless steel, a shape memory polymer, and a Co—Cr—Mo-based alloy.

10. The patient-specific spinal rod using 3D printing of claim 1, wherein at least one of a crosslink, a connector, or a spinal cord protector connecting two support portions in a transverse direction is integrally formed with the rod.

11. A pedicle fixation device, comprising:
a pedicle screw provided with a head portion configured to have an accommodation groove of a predetermined depth formed thereon, and a screw portion configured to be driven into a bottom surface of the head portion by a predetermined depth in a vertical direction of a pedicle, and serves as a fixing base for a damaged pedicle;
a rod seated in the accommodation groove, and configured to connect two or more pedicle screws to fix an angle and spacing of the pedicle; and
a fixing means fastened to the accommodation groove of the pedicle screw and configured to pressurize the rod to prevent the rod from moving,
wherein the rod is characterized in that information of a damaged spine is obtained through X-rays or computed tomography (CT) scans before surgery, an image is created by considering the setting of a coupling part of the rod that is combined with a pedicle screw inserted and fixed in a vertebral body to allow two or more vertebral bodies of the damaged spine to fuse and a bending shape according to a curve of a spine, and a metal material is melted and output by 3D printing to have an appearance corresponding to the created image, and comprises:
a rod-shaped coupling portion coupled with the pedicle screw; and
a support portion integrally formed with the coupling portion by extending in a longitudinal direction of the coupling portion, and configured to have a diameter or shape different from that of the coupling portion to serve as a support for holding the spine.
